(19) United States
(12) Patent Application Publication
(10) Pub. No.: US 2015/0073354 A1
(43) Pub. Date: Mar. 12, 2015

(54) MEASUREMENT INDICIA SYRINGE SLEEVE AND SYRINGE NEEDLE RETRACTION MECHANISM

(71) Applicant: Parenteral Technologies, LLC, Siesta Key, FL (US)

(72) Inventor: Michael A. Creaturo, Siesta Key, FL (US)

(21) Appl. No.: 14/483,403

(22) Filed: Sep. 11, 2014

Related U.S. Application Data
(60) Provisional application No. 61/876,385, filed on Sep. 11, 2013.
(60) Provisional application No. 61/879,823, filed on Sep. 19, 2013.

(51) Int. Cl. A61M 5/31

(52) U.S. Cl. CPC A61M 5/3129; A61M 2005/3126; A61M 2205/0238
USPC 604/189

(57) ABSTRACT

A measurement sleeve adapted for use with a medication syringe including an optically transparent body defining an axial passageway therethrough for receiving a barrel of the medication syringe therein, at least one lateral face through which the barrel is visible when the barrel is within the axial passageway, measurement indicia marked on the at least one lateral face along a portion of the length of the axial passageway, and a barrel lock at one end of the body configured to lock the barrel to the body to prevent relative axial movement therebetween.

### CROSS-REFERENCE TO RELATED APPLICATIONS

This application claims priority from U.S. Application No. 61/876,385 filed Sep. 11, 2013 and U.S. Application No. 61/879,823 filed Sep. 19, 2013, the entirety of which are incorporated by reference herein.

### TECHNICAL FIELD AND BACKGROUND OF THE INVENTION

The present invention relates generally to the field of medical syringes and accessories adapted for use with medical syringes, and more particularly, to a measurement indicia sleeve adapted for use with a medical syringe for the purpose of reducing or eliminating medical administered errors associated with injections, as well as a retraction mechanism for selectively concealing a syringe needle to prevent accidental sticks.

Medical administered errors occur in the healthcare field at an epidemic proportion, accounting for more deaths in a given year than deaths from motor vehicle accidents, breast cancer and AIDS. The U.S. Food and Drug Administration (FDA) reports that the most common errors are associated with medication injections, and particularly improper dosing, administering the wrong medication and the wrong route of administration. The FDA estimates that medication errors injure approximately 1.3 million people annually in the U.S. alone, with children being the most vulnerable due to low body mass. A low body mass makes one more sensitive to overdoses and underdoses, which result in ineffective treatment of the underlying condition, adverse reactions, illness and even death.

Medication errors occur both within and outside of the hospital setting. Errors stem from poor communication, misinterpreted handwriting, drug name confusion, incorrect drug strength, inaccurate dosage calculations, confusing drug labeling and packaging, lack of employee knowledge, and lack of patient understanding of drug directions. The resulting financial impact on the healthcare industry in the U.S. is staggering, and is estimated to exceed $100 billion annually.

Studies consistently support that the incorrect preparation, calculation and/or administration of injectable medications are among of the leading causes of medical administered errors, and continue to present a difficult procedural challenge to the healthcare industry. Errors are exacerbated, in large part, by user confusion caused by: (i) the complex process of preparing injectable medications for administration; (ii) the fact that different medications have similar names and labels; (iii) the need to administer different dosages of the same medications; (iii) the fact that supplies of the same medications often come measured in different unit strengths; (iv) the need to determine the size of the dosage to be administered to a patient on the basis of the patient's weight or other patient characteristic such as body surface area, age or degree of renal impairment; and (v) the long hours fast-pace and high-stress environment in which healthcare providers work.

Therefore, to help reduce the occurrence of medical administered errors, and in particular errors associated with medication injections, provided herein are syringes and accessories adapted for use with medication syringes that simplify the injection process and eliminate the need for dosage calculations, among other features.

### BRIEF SUMMARY OF THE INVENTION

It is an object of the invention to substantially eliminate some of the leading causes of medical administered errors associated with medication injections via syringe or other infusion or injection device.

It is another object of the invention to substantially eliminate medical administered errors associated with devices used for the parenteral administration of medication, diluent or other substances, devices used for the oral administration of medication, diluent or other substances, and devices used for the administration of medication, diluent or other substances by other means, any and all such devices collectively referred to herein as simply “injection devices.”

It is yet another object of the invention to substantially eliminate or facilitate user tasks associated with injection devices including, but not limited to, calculating the proper amount of medication or other substance to load into the injection device, loading of the proper amount, converting the measurement of the medication or other substance from one measurement index to another corresponding measurement index, determining proper dosage amount based on a patient characteristics or condition, and handling the injection device.

To achieve the foregoing and other objects and advantages, in one embodiment provided herein is a measurement indicia sleeve adapted for use with a medication syringe, the sleeve including an optically transparent body defining an axial passageway therethrough for receiving a barrel of the medication syringe therein and at least one lateral face through which the barrel is visible when the barrel is within the axial passageway, measurement indicia marked on the at least one lateral face along a portion of the length of the axial passageway, and a barrel lock at one end of the body configured to lock the barrel to the body to prevent relative axial movement therebetween.

In one aspect, the measurement indicia may include at least one of volumetric and weight calibrations.

In another aspect, the volumetric and weight calibrations may increase in measured quantity in the direction of the barrel lock.

In another aspect, the optically transparent body may define opposing lateral faces, at least one of which is marked with the measurement indicia.

In another aspect, one of the opposing lateral faces may be marked with medication dosage measurement indicia and the other of the opposing lateral faces may be marked with diluent measurement indicia.

In another aspect, the at least one lateral face may be marked with at least two sets of correlated measurement indicia selected from the group consisting of medication volumetric indicia, medication weight indicia, patient weight, and patient body surface area.

In another aspect, the barrel lock may be cylindrical and may define diametrically opposing slots at one end thereof adapted to receive and capture finger flanges of the barrel upon rotation of the barrel relative to the barrel lock.

In another aspect, the measurement sleeve may be a unitary body.

In another aspect, the at least one lateral face may be planar.

In another embodiment, the present invention provides a measurement sleeve adapted for use with a medication syringe including an optically transparent body defining an axial passageway therethrough and at least one lateral face marked with measurement indicia aligned along a portion of the length of the axial passageway, and a barrel lock at one end of the body for locking a barrel of the medication syringe to the measurement indicia sleeve to prevent relative axial movement therebetween.

In another aspect, the measurement indicia may include at least one of volumetric and weight calibrations, and the volumetric and weight calibrations may increase in measured quantity in the direction of the barrel lock.

In another aspect, the at least one lateral face may be marked with medication dosage measurement indicia, and may be marked with at least two sets of correlated measurement indicia selected from the group consisting of medication volumetric indicia, medication weight indicia, patient weight, and patient body surface area.

In another embodiment, provided herein is a syringe assembly including a syringe having a cylindrical barrel, a plunger formed for close fitting engagement within the barrel, and finger flanges on the barrel for manipulating the syringe, and a measurement sleeve adapted for use with the syringe and including an optically transparent body defining an axial passageway therethrough for receiving the barrel therein and at least one lateral face through which the barrel is visible when the barrel is within the axial passageway, the measurement sleeve including measurement indicia marked on the at least one lateral face along a portion of the length of the axial passageway and a barrel lock at one end of the body configured to lock the barrel to the body to prevent relative axial movement therebetween.

In a further aspect, the at least one lateral face may be marked with at least two sets of correlated measurement indicia selected from the group consisting of medication volumetric indicia, medication weight indicia, patient weight, and patient body surface area.

In a further aspect, the barrel lock may be cylindrical and may define diametrically opposing slots at one end thereof adapted to receive and capture the finger flanges upon rotation of the barrel relative to the barrel lock.

Embodiments of the invention can include one or more or any combination of the above features and configurations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 shows a first embodiment of a measurement sleeve having a syringe loaded therein;

FIG. 2 is an isometric view of the measurement sleeve of FIG. 1 presenting a first lateral side marked with measurement indicia;

FIG. 3 shows a syringe having a needle retraction mechanism loaded into the measurement sleeve;

FIG. 4 shows a syringe adapted for use with the measurement sleeve of FIG. 1;

FIGS. 5A and 5B are sequential views illustrating barrel and barrel lock engagement;

FIG. 6 is an isometric view of the measurement sleeve showing the opposing lateral side of the sleeve marked with diluent indicia;

FIG. 7 is a sectional view through the longitudinal length of the sleeve and syringe of FIG. 1;

FIG. 8 shows a second embodiment of a measurement sleeve having a syringe loaded therein;

FIG. 9 is front view of one lateral face of the measurement sleeve of FIG. 9;

FIG. 10 is an isometric view of the measurement sleeve of FIG. 9;

FIG. 11 shows a syringe adapted for use with the measurement sleeve of FIG. 10; and

FIG. 12 shows a multi-sided measurement sleeve.

### DETAILED DESCRIPTION OF THE INVENTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

Referring to the figures, the present invention provides various embodiments of measurement sleeve accessories adapted for use with conventional medication syringes, as well as syringes according to embodiments of the present invention, for the purpose of facilitating injections and reducing medical administered errors associated with injections, among other purposes. The various sleeves, syringes and combinations thereof may be provided as single components, combinations of components, and as components of calibrated kits further including medications. As used herein, the term “syringe” is intended to generically describe and encompass any form and type of injection device.

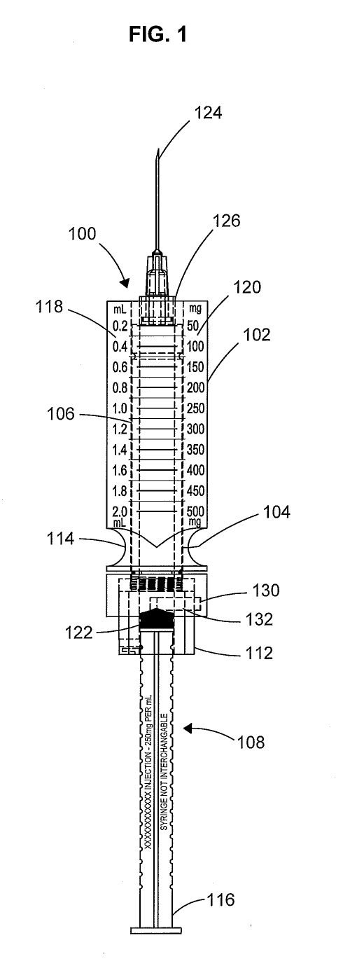

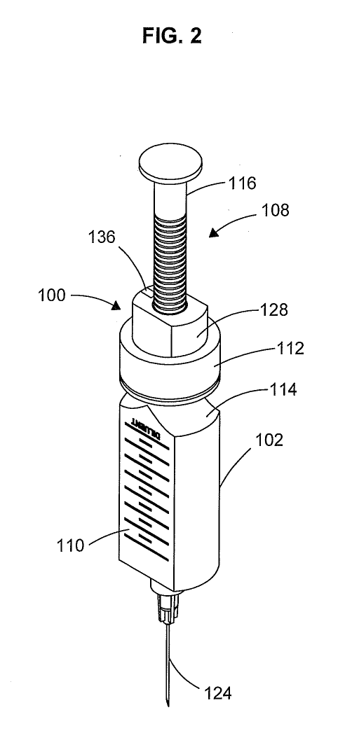

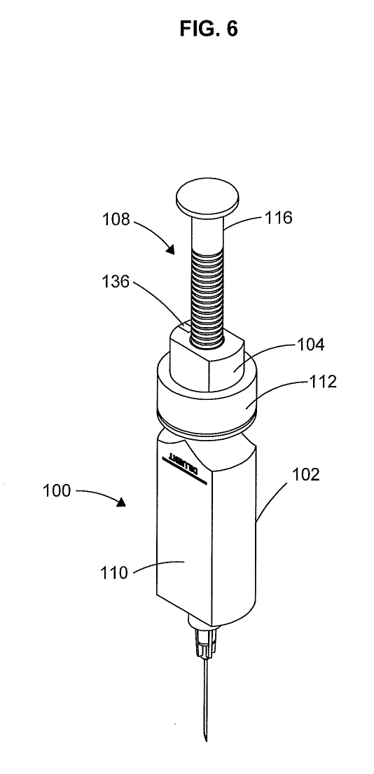

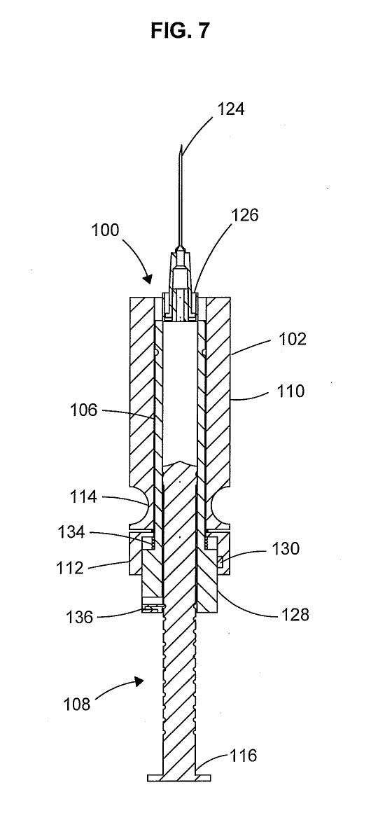

Referring to FIGS. 1-7, a first embodiment of a measurement sleeve adapted for use with a medication syringe is shown generally at reference numeral 100. The measurement sleeve 100, also referred to herein as the “sleeve,” generally includes an optically transparent rigid body 102 defining an axial passageway 104 therethrough for receiving a barrel 106 of a medication syringe 108 therein, preferably in relatively tight-fitting engagement to maintain a parallel relationship therebetween. The body 102 is formed to define at one end a first portion defining at least one lateral face 110, and at the opposing end a barrel lock 112. Interconnecting the first end and the barrel lock 112 is a reduced diameter portion 114, in one embodiment circumferentially recessed, to facilitating gripping the sleeve 100 to actuate the syringe plunger 116.

The body 102 is optically transparent or translucent such that the syringe, and specifically the syringe barrel 106 and liquid therein, is visible to the eye through the at least one lateral face 110 when the syringe is loaded in the sleeve 100. The body 102 may be constructed from various materials including, but not limited to, plastics, glass and acrylic. As shown, the body 102 defines two lateral faces 110 on opposite sides of the body, connected along their sides by arcuate portions to facilitate gripping and ergonomics. It is envisioned that the body 102 may include one lateral face, two lateral faces, three lateral faces, four lateral faces, and more than four lateral faces. In the case of two lateral faces 110, the two faces may be parallel. In the case of three lateral faces 110, two of the faces may be parallel and the third may be perpendicular to the two parallel faces, or the three may have a triangular relationship. In the case of four lateral faces, the faces may be arranged into two pairs of parallel faces with a perpendicular relationship between the pairs. The at least one lateral face 110 may be planar, substantially planar, may have a slight curvature, or may have a shape that is a combination of planar and curved. In a preferred embodiment, the at least one lateral face 110 is planar to facilitate reading marked indicia thereon and eliminate optical distortion associated with curved surfaces.

The at least one lateral face 110 is marked with measurement indicia along at least a portion of the length thereof coincident with a portion of the length of the axial passageway 104. The measurement indicia, shown at reference numerals 118 and 120, indicate one or more of medication and/or diluent volume, weight, patient characteristic, purity, etc., as discussed in detail below. The markings on the at least one lateral face 110, preferably in the form of graduations increasing in measured quantity in the direction of the barrel lock 112, enhance, override or take the place of any graduations marked on the barrel 106. In this regard, the sleeve 100 itself serves as the measurement guide for determining the amount of medication and/or diluent in the syringe 108.

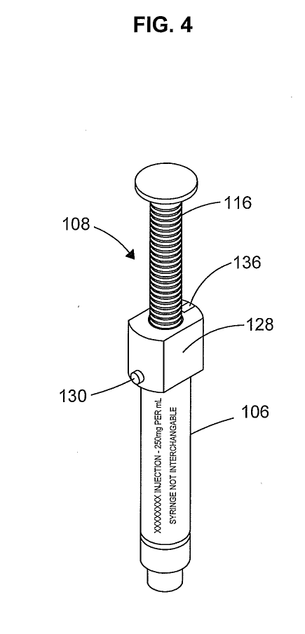

The sleeve 100, and particularly the barrel lock 112, may be configured to accommodate a conventional syringe or a syringe according to an embodiment of the present invention. As shown, the syringe 108 is generally of the type including a barrel 106, a plunger 116 arranged to reciprocate within the barrel, a seal 122 provided on the end of the plunger, and a needle 124 configured to attach to one end of the barrel by way of a luer lock 126. A collar 128 is provided at the opposite end of the barrel 106 from the needle. As best shown in FIG. 4, the collar 128 includes a protrusion 130, and as shown a single protrusion, extending laterally therefrom to so as to engage within a slot defined in the barrel lock 112 of the sleeve 100 to lock the syringe to the sleeve.

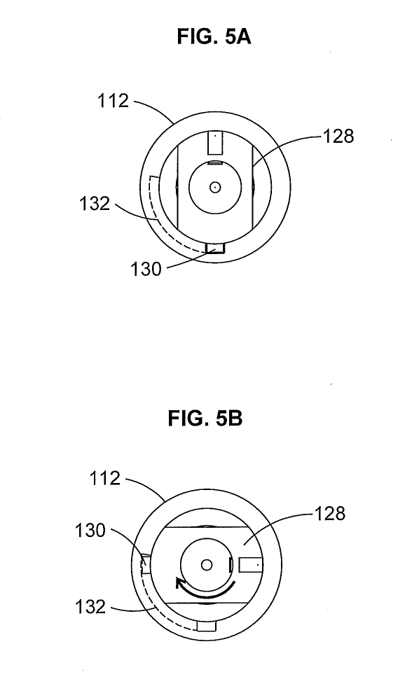

Referring specifically to FIGS. 5A and 5B, locking the syringe 108 to the sleeve 100, and in particular axially locking the barrel 106 relative to the sleeve, is achieved by engaging the protrusion 130 within a slot 132 defined in one end of the barrel lock 112. The slot 132 first extends a short length axially in a direction toward the at least one lateral face end of the body 102, then gradually or abruptly turns 90 degrees and follows along a portion of the inner circumference of the barrel lock 112. With such a slot path, a portion of the collar 128 can be advanced partway into the barrel lock 112 with the protrusion 130 aligned with the axial portion of the slot 132 as shown in FIG. 5A, and then the barrel 106 turned about 90 degrees relative to the barrel lock such that the protrusion moves to the far end of the slot as shown in FIG. 5B. In this arrangement, when the protrusion 130 is at the far end of the slot 132, relative axial movement between the barrel and the sleeve 100 is prevented and the two cannot be pulled apart axially. Thus, the alignment of the at least one lateral face 110 with the syringe barrel 106 can be counted on when relying on the measurement indicia on the sleeve 100.

One or more of the syringe barrel 106 and plunger 116 may be marked with a medication name, type, volumetric information, weight information, volume to weight correlation, maximum dosage, etc. as shown in FIGS. 1 and 4.

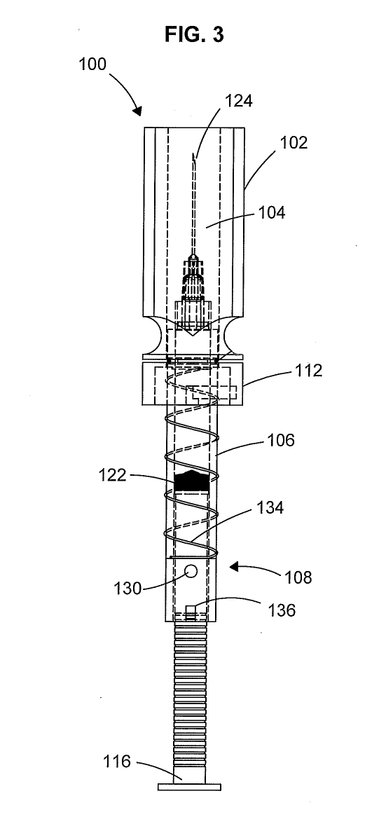

Referring specifically to FIG. 3, syringe 108 may include a needle retraction mechanism for selectively retracting the needle 124 within the axial passageway 104 of the sleeve 100 as desired to prevent unintentional needle sticks. The retraction mechanism is configured to allow the needle to be retracted at the sole discretion of the user at any time, regardless of the amount of the contents within the syringe. The protrusion 130 may have dual functionality, not only locking the barrel 106 axially relative to the sleeve 100 in use, but also locking the barrel in the proper position within the sleeve such that the needle extends beyond the end of the sleeve. A biasing member, such as helical spring 134, is seated and captured between the barrel lock 112 and the collar 128. In this arrangement, the helical spring 134, which is wrapped around the barrel 106, biases the barrel in the direction axially away from the barrel lock 112 such that when the protrusion 130 is disengaged from the barrel lock the needle 124 is concealed within the sleeve body 102. The helical spring 134 is loaded when the barrel 106 is axially advanced into the sleeve 102.

The plunger 116 may also be locked axially relative to the barrel by engaging a laterally sliding lock 136 on the collar 128 in the direction of the plunger, thereby engaging ribbing along the length of the plunger.

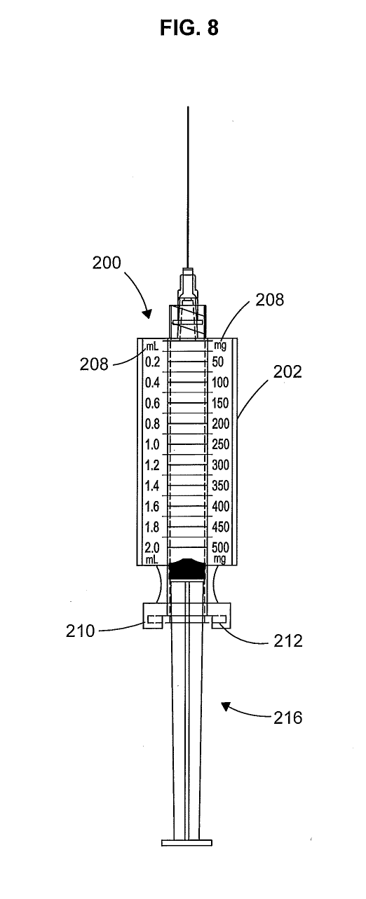

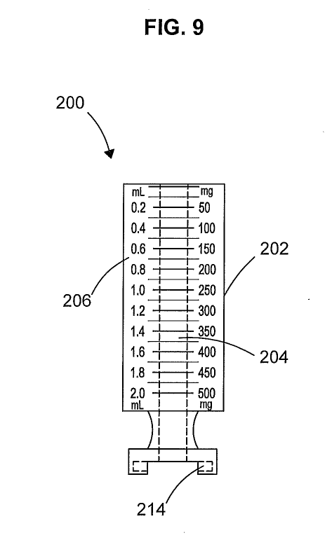

Referring to FIGS. 8-10, a second embodiment of a measurement sleeve adapted for use with a medication syringe is shown generally at reference numeral 200. Like sleeve 100, sleeve 200 generally includes an optically transparent body 202 formed to define an axial passageway 204 therethrough, at least one lateral face 206 marked with measurement indicia 208, a barrel lock 210, and an interconnecting portion interconnecting the lateral face carrying portion with the barrel lock to facilitate gripping and plunger actuation.

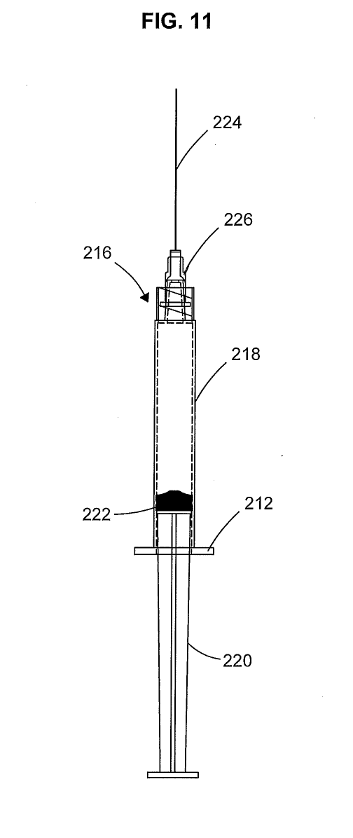

The difference between sleeve 200 and sleeve 100 is primarily in the configuration of the barrel lock 210. Specifically, while barrel lock 112 is configured to receive and engage with a syringe including a single lateral protrusion, barrel lock 210 is configured to receive and engage with a syringe having diametrically opposing and laterally extending finger flanges. One example of such a syringe including finger flanges is shown in FIG. 11, wherein the finger flanges are indicated at reference numeral 212.

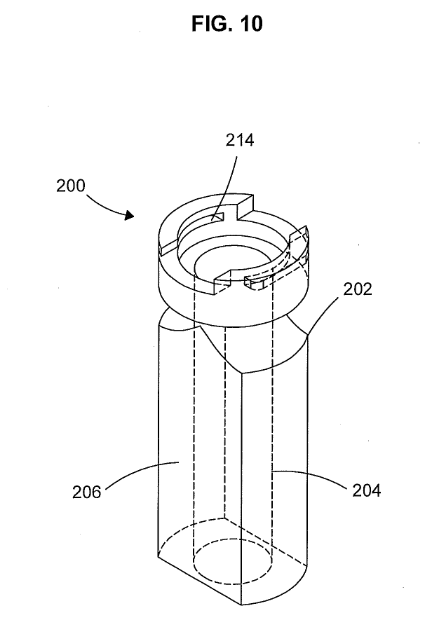

Referring specifically to FIG. 10, the barrel lock 210 is generally cylindrical and defines diametrically opposing slots 214 at one end thereof adapted to receive the finger flanges 212 of the barrel upon rotation of the barrel relative to the barrel lock. As shown, the slots 214 are formed by a material void following a portion of the circumference of the barrel lock 210. Entry into the respective slots is in opposite directions. To engage the finger flanges 212 within their respective slot 214, the finger flanges are first seated on the top of the barrel lock 210 against the flats between slots. The barrel is then rotated in a first direction, for example clockwise, about 90 degrees to fully engage the finger flanges 212 within their respective slot 214. When engaged therein, such as fully engaged, relative axial movement between the barrel and the sleeve 200 is prevented, ensuring alignment between the at least one lateral face 206 and the syringe barrel. The syringe is removed from the sleeve 200 by rotating the barrel relative to the sleeve in the direction opposite the engagement direction, for example counterclockwise, to clear the finger flanges from their respective slots, allowing the barrel to be withdrawn (i.e., pulled axially) from the sleeve 200.

Referring to FIG. 11, syringe 216 generally includes including a cylindrical tubular barrel 218, a plunger 220 concentrically arranged within the barrel and configured to reciprocate therein, an elastomeric seal 222 provided at one end of the plunger, and a needle 224 configured to attach to one end of the barrel by way of a Luer lock 226.

In a further embodiment, provided herein is a syringe assembly including a syringe comprising a cylindrical barrel and a plunger configured to reciprocate within the barrel, and a measurement sleeve comprising an optically transparent body defining an axial passageway therethrough and at least one lateral face through which the barrel is visible when the barrel is within the axial passageway, the measurement sleeve including measurement indicia marked on the at least one lateral face along a portion of the length of the axial passageway.

The measurement sleeve may further include a barrel lock at one end of the body configured to lock the barrel to the body to prevent relative axial movement therebetween. In another embodiment, the cylindrical barrel and the measurement sleeve may be integrally formed, therefore obviating the need for locking the component together.

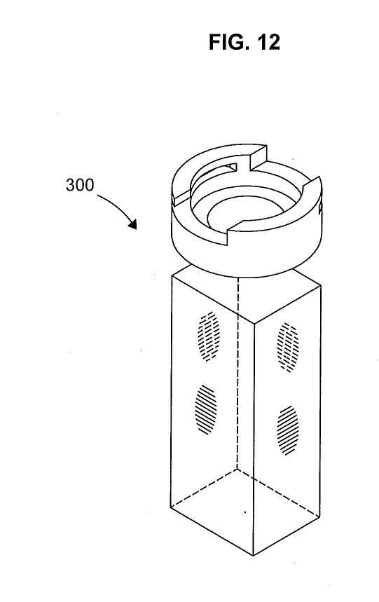

Referring to FIG. 12, a further embodiment of a measurement sleeve is shown at reference numeral 300. The measurement sleeve 300 includes a multi-sided body and barrel lock wherein one or more of the lateral faces are marked with any of the measurement indicia described herein, as well as medication information, diluent information, other visual markings or textures, etc. The measurement sleeve 300 is shown in FIG. 12 without the axial passageway for clarity, however it should be understood that the sleeve 300 would include such a passageway as described above. Pairs of opposing lateral faces may be marked with the same indicia or different indicia, which may be different from the indicia markings on the adjacent sides.

In the above embodiments, one of the opposing lateral faces may be marked with medication dosage measurement indicia while the other of the opposing lateral faces may be marked with diluent measurement indicia. In another aspect, the at least one lateral face may be marked with at least two sets of correlated measurement indicia selected from medication volumetric indicia, medication weight indicia, patient weight, patient body surface area, purity, other patient characteristics, etc.

The at least one lateral face allows the barrel to be visible through the measurement sleeve, which inherently allows the medication in the syringe to also be visible through the barrel and the sleeve. In this regard, the user is able to see the amount of liquid in the syringe and use the calibrated markings on the measurement sleeve to ensure the correct dosage. Each lateral face may be marked with one or more sets of measurement indicia, thereby enabling the user to measure the amount of medication or other substance in the units of measurement of each such measurement indicia. For example, if a set of measurement indicia were calibrated to a specific medication in milligrams, the lateral face bearing such indicia would permit the user to measure the amount of the medication or other substance in the syringe in milligrams. The at least one lateral face enables the user to make a more precise and reliable measurement of the volume of the medication or other substance in the syringe than is possible using only the syringe itself, which typically has a curved surface.

In each of the above embodiments, the at least one lateral face may include more than one set of measurement indicia. Each set of measurement indicia may measure the volume of the medication or other substance in the syringe in accordance with a different measurement scale or criterion. The measurement indicia scales may appear in juxtaposition to one another so that the user can simultaneously measure the contents of the syringe in more than one measurement scale or criterion without having to perform calculations to convert or correlate a measurement in one set of units or criteria to a measurement in another set of units or criterion. For example, one set of measurement indicia may be in cubic centimeters/milliliters, while another may display a specific volumetric dosage in milligrams, micrograms or international units of the medication or substance contained in the syringe.

The measurement indicia may display the conversion from volumetric measurement indicia to a measurement based on a specific patient feature. For example, the lateral face may show the conversion of an indicia in cubic centimeters, milliliters, international units, milligrams or micrograms into a recommended dosage measurement based on patient weight or patient body surface area indicia. In further examples, the measurement indicia displayed on the sleeve may be calibrated from the manufacturer's suggested dosage schedule of a medication or other substance, whereby the first indicia may indicate the volumetric dosage in milligrams correlating to the volume of the medication in milliliters, and another indicia displaying the corresponding reduced and/or minimum recommended dosage (e.g., for pediatrics or renal impaired patients), and yet another indicia for the maximum dosage correlated according to patient weight, patient body surface area or other patient characteristic.

One or more of the measurement indicia may display the manufacturer's suggested dosage schedule of a medication or other substance whereby a first indicia may measure the contents of the syringe in volumetric measurement units, while a second set of indicia may measure the volume of the contents as it relates to the recommended dosage for a particular patient feature (e.g., age, weight and/or particular ailment or diagnosis) and/or the maximum or minimum dosage of the medication or other substance calibrated to a patient feature, a particular aliment and/or particular characteristics of the medication or other substance for which the syringe is designed.

Obviating the need for user calculations and conversions helps to prevent errors in the determination of the proper dosage, the extraction of the proper dosage, the loading of the proper dosage, the adjustment of the size of the dosage, and the administration of the proper dosage, while also obviating all procedures and calculations to accomplish the correct preparation and administration of the medication or other substance for specific dosage, measurement, patient weight, patient body surface area, other patient characteristics or other patient and/or specific targeted dosage.

In certain embodiments, the measurement indicia scales may appear in juxtaposition to one another so that the user can simultaneously measure the contents of the syringe or other instrument in more than one set of measurement units, without the need to perform conversion calculations. Multiple measurement indicia may appear on the same lateral face or each such set of indicia may appear on a separate lateral face. For example, in the case of a four-sided sleeve as shown in FIG. 12, one or more sets of measurement indicia may appear on the “front” face, one or more sets of indicia may appear on the “back” face, one or more sets of indicia may appear on the “right” face, and one or more sets of indicia may appear on the “left” face.

As compared to a conventional round-barreled syringe, the lateral face provides significantly more surface area that can be used to display additional information about the medication or other substance contained in the syringe, the administration thereof, and the recommended dosage thereof based on characteristics of the patient and/or the patient's illness and/or the medication or other substance contained therein. In specific embodiments, the surface area of each lateral face may be from 2 cm² to 20 cm², from 5 cm² to 15 cm², from 5 cm² to 10 cm², from 10 cm² to 20 cm², from 10 cm² to 15 cm², any range between 2 and 20 cm², and more than 20 cm².

Any of the sleeve and/or syringe components described and shown herein may include printed information identifying a medication or other substance. For example, one or more of the lateral faces, the plunger and the barrel may bear such information. Information may include, but is not limited to, medication name, medication strength, medication dosage-to-weight ratio and/or combinations thereof.

Further embodiments of the invention contemplate kits comprised of the devices and systems described herein and/or medications and/or diluents and/or other substances, wherein one or more of the devices may be pre-calibrated with indicia to deliver the correct amount of diluent to reconstitute the medication and calibrated to the medication or other substance with one or more calibrated measurement indicia and/or volumetric designs, either pre-filled, or ready-to-fill, that specifically correlate volumetric measurement or other characteristic of the medication or other substance to one or more other measurements and/or dosages or patient weight or other characteristic. For example, a syringe with one indicia pre-calibrated for milligrams or other dosage or a syringe pre-calibrated to display a specific amount of medication in kilograms or other patient characteristic indicia.

Correlation measurements may include volume measured in, for example, milliliters or cubic centimeters, to the dosage strength measured in, for example, micrograms, milligrams, or international units, or to other activity or characteristic. In this regard, the user does not need to perform any calculations to determine the proper corresponding volumetric dosage or diluent amount. The measurement indicia may further correspond volume-to-activity, potency, international units, assay, milligrams, micrograms, or other characteristic of the medication or other substance, for example, volume shown in milliliters/cubic centimeters of a medication to a dosage size measured in micrograms, milligrams, or international units.

In a further embodiment, the lateral faces may display and correlate milliliters/cubic centimeters to milligrams, micrograms, international units or some other potency on the front face, the left panel may display the name, dosage or other data regarding the contents of the injection, the right face may display diluent data and/or indicia for the correct amount of diluent required for the reconstitution, and the back face may display recommended minimum and maximum dosage indicia according to a patient characteristic, such as weight. In this regard, the user is provided with four calibrated indicia measurements for one medication or substance, obviating the need for calculations.

One or more of the components described herein may further bear indicia for the recommended amount and/or volume of a particular diluent that may be indicated for the medication or substance designed and/or calibrated for, and may also bear indicia and additional data regarding a diluent. For example, such additional data may include instructions for the reconstitution of the medication and/or the amount of the medication or diluent, or the name of the medication or diluent. Still further, components may bear indicia and data regarding a particular dosage and/or data such as a schedule for the administration of the medication or substance and/or the administration of the medication to patients with specific characteristics. For example, such data may include a dosage schedule for the elderly, pediatric patients, patients with certain creatinine levels and renal impaired patients, among others.

The sleeves described herein may be customized to be adapted for use with conventional syringes, for example, 1 mL, 2 mL, 3 ml, 5 ml, 10 mL, 20 mL, 50 ml and 100 mL syringes, among others.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. A measurement sleeve adapted for use with a medication syringe, comprising:
an optically transparent body defining an axial passageway therethrough for receiving a barrel of the medication syringe therein and at least one lateral face through which the barrel is visible when the barrel is within the axial passageway;
measurement indicia marked on the at least one lateral face along a portion of the length of the axial passageway; and
a barrel lock at one end of the body configured to lock the barrel to the body to prevent relative axial movement therebetween.

2. The measurement sleeve of claim 1, wherein the measurement indicia comprises at least one of volumetric and weight calibrations, and wherein the volumetric and weight calibrations increase in measured quantity in the direction of the barrel lock.

3. The measurement sleeve of claim 1, wherein the optically transparent body defines opposing lateral faces, at least one of which is marked with the measurement indicia.

4. The measurement sleeve of claim 3, wherein one of the opposing lateral faces is marked with medication dosage measurement indicia and the other of the opposing lateral faces is marked with diluent measurement indicia.

5. The measurement sleeve of claim 1, wherein the at least one lateral face is marked with at least two sets of correlated measurement indicia selected from the group consisting of medication volumetric indicia, medication weight indicia, patient weight, and patient body surface area.

6. The measurement sleeve of claim 1, wherein the barrel lock is cylindrical and defines diametrically opposing slots at one end thereof adapted to receive finger flanges of the barrel upon rotation of the barrel relative to the barrel lock.

7. The measurement sleeve of claim 1, wherein the barrel lock is cylindrical and defines a slot accessible through one end thereof adapted to receive a protrusion on the barrel to lock the barrel relative to the barrel lock upon rotation of the barrel relative to the barrel lock.

8. The measurement sleeve of claim 1, wherein the measurement sleeve is a unitary body and the at least one lateral face is planar.

9. A measurement sleeve adapted for use with a medication syringe, comprising:
an optically transparent body defining an axial passageway therethrough and at least one lateral face marked with measurement indicia aligned along a portion of the length of the axial passageway; and
a barrel lock at one end of the body for locking a barrel of the medication syringe to the measurement indicia sleeve to prevent relative axial movement therebetween.

10. The measurement sleeve of claim 9, wherein the measurement indicia comprises at least one of volumetric and weight calibrations.

11. The measurement sleeve of claim 9, wherein the optically transparent body defines opposing lateral faces, at least one of which is marked with the measurement indicia.

12. The measurement sleeve of claim 11, wherein one of the opposing lateral faces is marked with medication dosage measurement indicia and the other of the opposing lateral faces is marked with diluent measurement indicia.

13. The measurement sleeve of claim 9, wherein the at least one lateral face is marked with at least two sets of correlated measurement indicia selected from the group consisting of medication volumetric indicia, medication weight indicia, patient weight, and patient body surface area.

14. The measurement sleeve of claim 9, wherein the barrel lock defines diametrically opposing slots at one end thereof adapted to receive and capture finger flanges of the barrel upon rotation of the barrel relative to the barrel lock.

15. The measurement sleeve of claim 9, wherein the barrel lock defines a slot accessible through one end thereof adapted to receive a protrusion on the barrel to lock the barrel relative to the barrel lock upon rotation of the barrel relative to the barrel lock.

16. The measurement sleeve of claim 9, wherein the measurement sleeve is a unitary body and wherein the at least one lateral face is planar.

17. A syringe assembly, comprising:
a syringe comprising a cylindrical barrel and a plunger configured to reciprocate within the barrel; and
a measurement sleeve comprising an optically transparent body defining an axial passageway therethrough and at least one lateral face through which the barrel is visible when the barrel is within the axial passageway, the measurement sleeve including measurement indicia marked on the at least one lateral face along a portion of the length of the axial passageway.

18. The syringe assembly of claim 17, further comprising a barrel lock at one end of the body configured to lock the barrel to the body to prevent relative axial movement therebetween.

19. The syringe assembly of claim 17, wherein the at least one lateral face is marked with at least two sets of correlated measurement indicia selected from the group consisting of medication volumetric indicia, medication weight indicia, patient weight, and patient body surface area.

20. The syringe assembly of claim 17, wherein the cylindrical barrel and the measurement sleeve are integrally formed.
